(19) United States
(12) Patent Application Publication
(10) Pub. No.: US 2016/0227869 A1
(43) Pub. Date: Aug. 11, 2016

(54) REVOLUTIONZ THE ULTIMATE SHOE

(71) Applicant: Eddie James WILLIAMS, JR., Queen Creek, AZ (US)

(72) Inventor: Eddie James WILLIAMS, JR., Queen Creek, AZ (US)

(21) Appl. No.: 14/810,076

(22) Filed: Jul. 27, 2015

Related U.S. Application Data
(60) Provisional application No. 62/114,946, filed on Feb. 11, 2015.

(51) Int. Cl. A43B 3/00; A61B 5/024; A61B 5/00; A61B 5/103; A43B 5/06; A63B 24/00

(52) U.S. Cl. CPC A43B 3/001; A43B 5/06; A63B 24/0062; A61B 5/6807; A61B 5/1038; A61B 5/743; A61B 5/0022; A61B 5/02438

(57) ABSTRACT

A workout system is provided. The workout system includes a pair of shoes and a handheld device. The pair of shoes may include an electronic chip. The electronic chip may have an impact detector to detect the amount of steps taken by the shoes. The electronic chip may further include a wireless transceiver. The handheld device includes a processor, a wireless transceiver, a global positioning system (GPS), and a monitor. The transceiver of the handheld device exchanges data with the transceiver of the shoes. The processor processes data from the transceiver and the GPS and displays the data on the monitor. The monitor may display at least one of an amount of impacts detected by the impact detector, a location of the handheld device, a distance traveled of the handheld device, and a speed of the handheld device.

### CROSS-REFERENCE TO RELATED APPLICATION

This application claims the benefit of priority of U.S. provisional application No. 62/114,946, filed Feb. 11, 2015, the contents of which are herein incorporated by reference.

### BACKGROUND OF THE INVENTION

The present invention relates to a workout system and, more particularly, to a workout system including an electronic chip within a pair of shoes linked with a handheld device.

Physical exercise is any bodily activity that enhances or maintains physical fitness and overall health and wellness. It is performed for various reasons, including strengthening muscles and the cardiovascular system, honing athletic skills, weight loss or maintenance, and merely enjoyment. Frequent and regular physical exercise boosts the immune system and helps prevent the “disease of affluence” such as heart disease, cardiovascular disease, Type 2 diabetes, and obesity. It may also help prevent depression, help to promote or maintain positive self-esteem, improve mental health generally, and can augment an individual's sex appeal or body image, which has been found to be linked with higher levels of self-esteem.

As can be seen, there is a need for interactive devices that may be used for exercise.

### SUMMARY OF THE INVENTION

In one aspect of the present invention, a workout system comprises: a pair of shoes, wherein at least one of the pair of shoes comprises an electronic chip comprising an impact detector and a wireless transceiver; and a handheld device comprising a processor, a wireless transceiver, a global positioning system, and a monitor, wherein the wireless transceiver of the handheld device exchanges data with the wireless transceiver of the pair of shoes wherein the monitor displays at least one of an amount of impacts detected by the impact detector, a location of the handheld device, a distance traveled of the handheld device, and a speed of the handheld device.

These and other features, aspects and advantages of the present invention will become better understood with reference to the following drawings, description and claims.

### BRIEF DESCRIPTION OF THE DRAWINGS

FIG. 1 is a schematic view of an embodiment of the present invention;

FIG. 2 is a flow chart of the general use of the present invention;

FIG. 3 is a schematic view of the monitor of FIG. 1 displaying a GPS mode;

FIG. 4 is a flow chart of the GPS function of FIG. 3;

FIG. 5 is a schematic view of the monitor of FIG. 1 displaying a music mode;

FIG. 6 is a flow chart of the music function of FIG. 6;

FIG. 7 is a schematic view of the monitor of FIG. 1 displaying safety lights mode;

FIG. 8 is a flow chart of the safety lights function of FIG. 7;

FIG. 9 is a schematic view of the monitor of FIG. 1 displaying workout programs mode;

FIG. 10 is a flow chart of the workout programs function of FIG. 9;

FIG. 11 is a schematic view of the monitor of FIG. 1 displaying distress signal mode;

FIG. 12 is a flow chart of the distress signal function of FIG. 11;

FIG. 13 is a schematic view of the monitor of FIG. 1 displaying biking mode;

FIG. 14 is a flow chart of the biking function of FIG. 13;

FIG. 15 is a schematic view of the monitor of FIG. 1 displaying texting mode;

FIG. 16 is a flow chart of the texting function of FIG. 15;

FIG. 17 is a schematic view of the monitor of FIG. 1 displaying application store mode;

FIG. 18 is a flow chart of the application store function of FIG. 17;

FIG. 19 is a schematic view of the monitor of FIG. 1 displaying heart rate mode; and

FIG. 20 is a flow chart of the heart rate function of FIG. 19.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a workout system including athletic shoes equipped with lights powered by batteries for safe night time running, and an electronic chip that contains information that may be fed into a hand held monitor. The monitor may control the lights and can transmit music or other information to wireless ear buds for the exerciser's enjoyment.

The electronic chip and the night lights may be built inside of the shoe. The hand held monitor allows viewing the exercisers' workout progress by time and distance. The present invention may include a Global Position System (GPS), night time workouts, and the exerciser may download and listen to music with the wireless ear buds. The shoe transmits the information and saves it to the monitor. The features may be activated from the workout monitor.

The system has batteries inside the shoe and has an informational chip inside of the soul. The chip may transmit the information to the monitor. The monitor has the GPS system and workout programs. The monitor also may include enough memory to save workout progress and download music. To use the present invention, the exerciser turns on the monitor, puts on the shoes, calibrates the GPS system and sets the features the user would like to use.

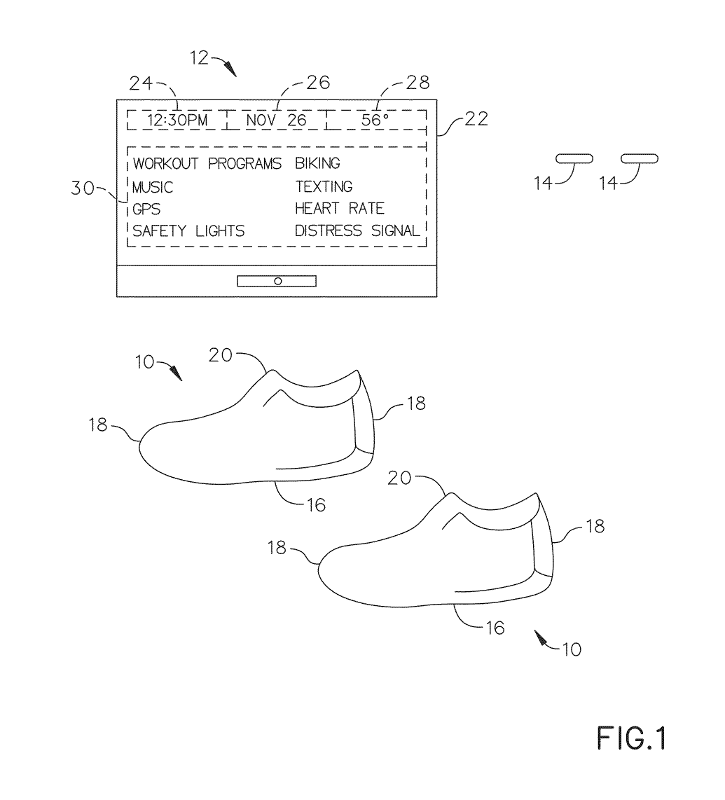

Referring to FIGS. 1 through 20, the present invention includes a workout system. The workout system includes a pair of shoes 10 and a handheld device 12. The pair of shoes 10 may include an electronic chip 16 and batteries 20. The electronic chip 16 may have an impact detector to monitor the amount of steps taken within the shoes. The electronic chip 16 may further include a wireless transceiver. The handheld device 12 includes a processor, a wireless transceiver, a global positioning system (GPS), and a monitor 22. The transceiver of the handheld device 12 exchanges data with the transceiver of the shoes 10. The processor processes data from the transceiver and the GPS and displays the data on the monitor 22. The monitor 22 may display at least one of an amount of impacts detected by the impact detector, a location of the handheld device 12, a distance traveled of the handheld device 12, and a speed of the handheld device 12.

The present invention may include additional features. In certain embodiments, the handheld device 12 may include a memory. The memory may store music data. In such embodiments, the system of the present invention may include an output, and headphones 14 connectable to the output. The headphones 14 may be wireless and the output may be the wireless transceiver. The handheld device 12 may further include a heart rate monitor. The shoes 10 of the present invention may further include safety lights 18.

As illustrated in FIG. 1, the monitor 22 may project a selection screen 30 in which a user may select a category of workout programs, music, GPS, safety lights, biking, texting, heart rate, distress signal, and the like. The monitor 22 may further project the time 24, the date 26 and the temperature 28. In certain embodiments, the time, distance, and other alerts may also be emitted through the headphones 14.

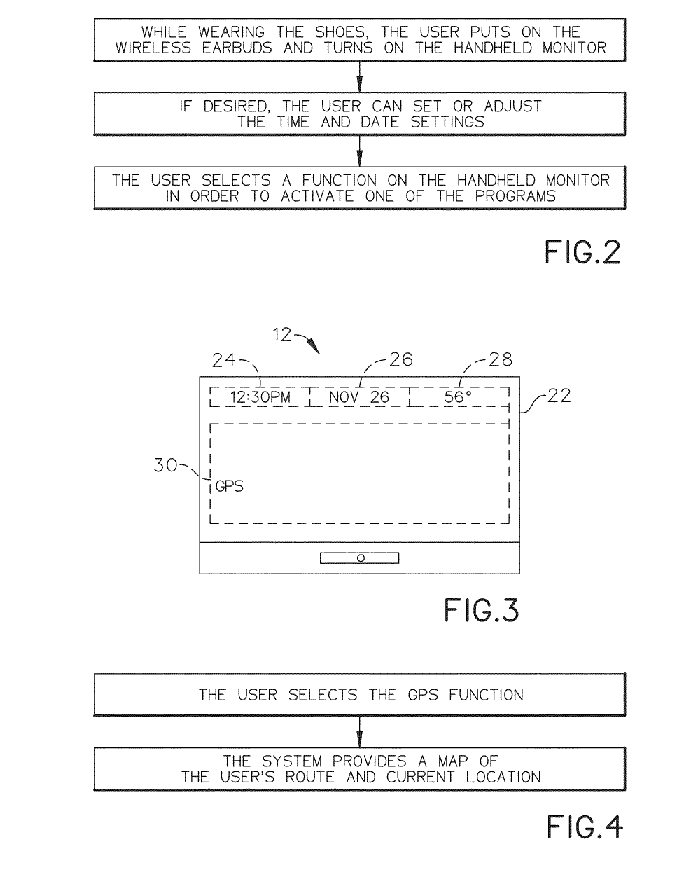

FIGS. 2 through 20 illustrate the use of the present invention. While wearing the shoes 10, the user may wear the headphones 14, such as ear buds, and turn on the handheld device 12. The user may then select a function on the handheld monitor 12 from the selection screen 30 to activate one of the programs. As illustrated in FIG. 3, the user may select the GPS function. The monitor 22 displays a map of the user's route and current location.

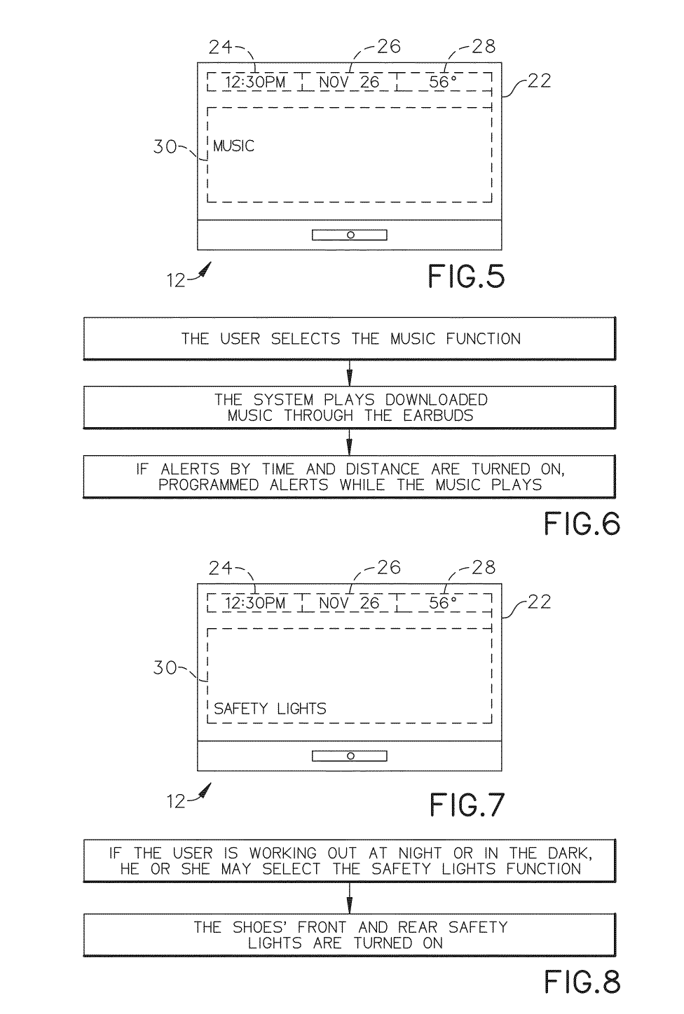

As illustrated in FIGS. 5 and 6, the user may select music from the selection screen 30. A list of music stored on the memory may be monitored for the user. The user may select the music to play on the headphones 14. Alternatively, the user may select shuffle in which the music stored on the memory may shuffle and play on the headphones 14 randomly. In certain embodiments, if the alerts are to be emitted through the headphones 14, the music volume may be lowered and the alerts may be emitted.

As illustrated in FIGS. 7 and 8, the safety lights 18 may be activated using the handheld device 12. If the user is working out at night or in the dark, the user may select the safety lights function from the selection screen 30. A command is sent from the transceiver of the handheld device to the transceiver of the shoes 10, and the safety lights 18 are turned on. The lights 18 may be located on the front and the rear of the shoes 10.

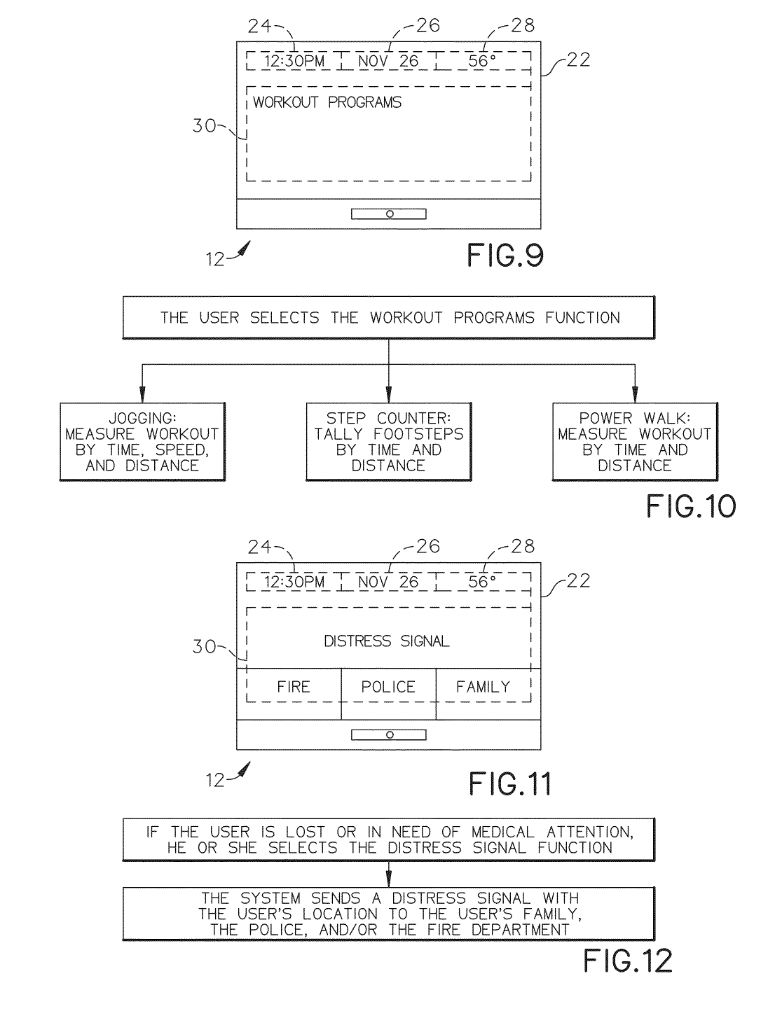

As illustrated in FIGS. 9 and 10, the user may select a workout program from the selection screen 10. Once the user has selected the workout program, the monitor 22 may project a plurality of workout routines for the user to choose from. For example, the user may select jogging, step counter, and power walk. Jogging may activate the measurement of the workout by time, speed and distance. The step counter may tally the footsteps by time and distance. The power walk may measure workout by time and distance.

As illustrated in FIGS. 11 and 12, the user may select a distress signal from the selection screen 30. If the user is lost or in need of medical attention, the user may select the distress signal function. The user may be given the option to send the distress signal to a fire department, the police, or to family. The system sends a distress signal with the user's location to the user's family, the police and/or the fire department.

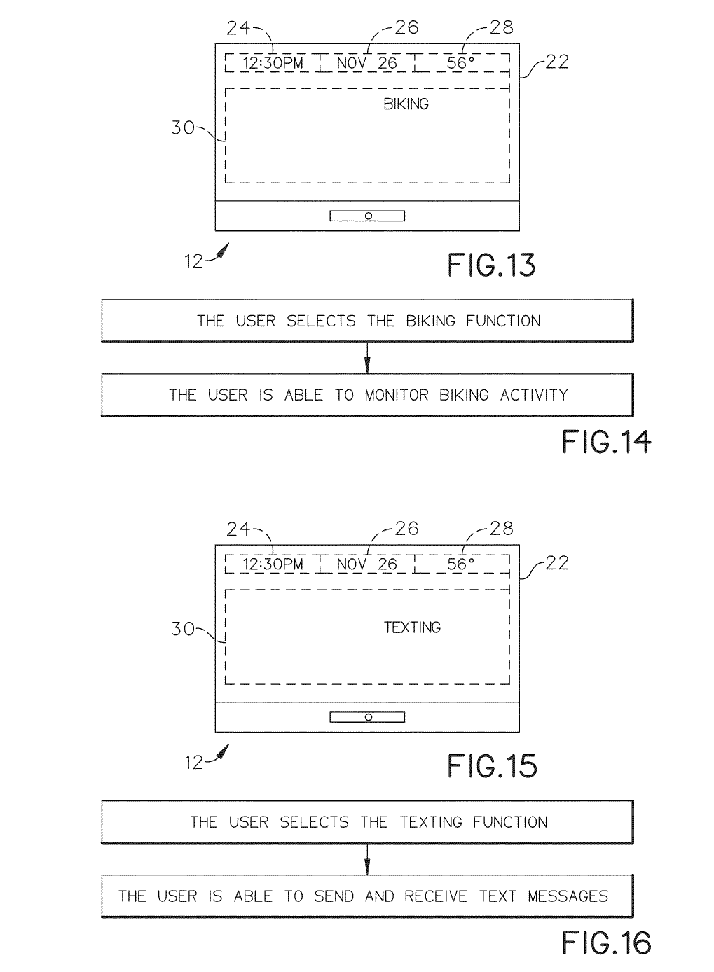

As illustrated in FIGS. 13 and 14, the user may select a biking function from the selection screen 30. In such embodiments, the user may monitor biking activity, such as speed, distance, location and the like.

As illustrated in FIGS. 15 and 16, the user may select a texting function from the selection screen 30. The user is able to send and receive text messages to other phone numbers and email addresses.

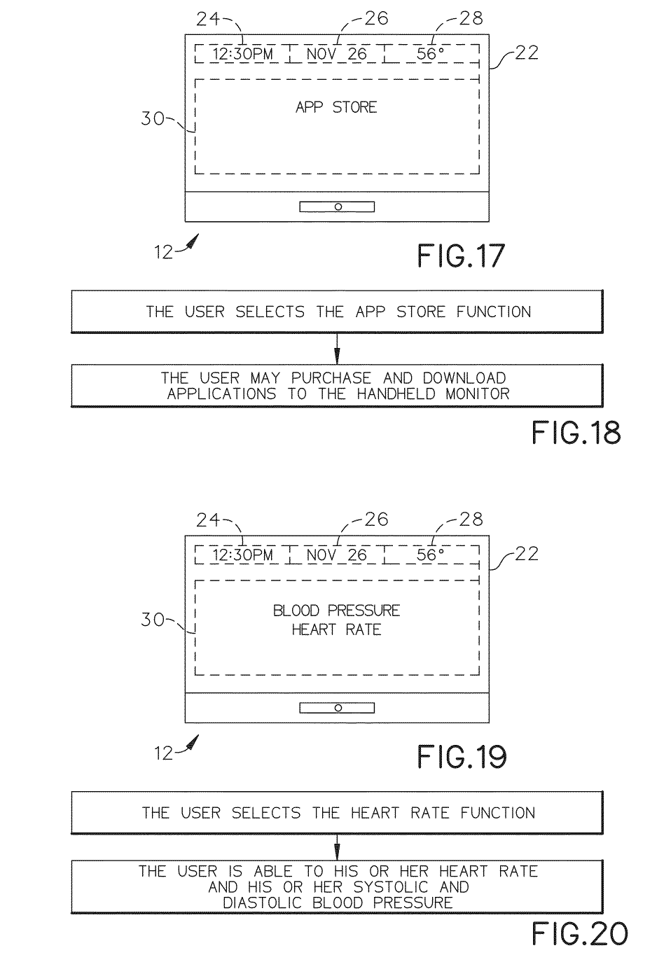

As illustrated in FIGS. 17 and 18, the user may select an app store function from the selection screen 30. In such embodiments, the user may purchase and download applications to the handheld device 10. Therefore, the handheld device 10 may include a wireless connection to the internet.

As illustrated in FIGS. 19 and 20, the user may select the heart rate function from the selection screen 30. In such embodiments, the user is able to check his or her heart rate and systolic and diastolic blood pressure. The readings may be displayed on the monitor 22.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A workout system comprising:
a pair of shoes, wherein at least one of the pair of shoes comprises an electronic chip comprising an impact detector and a wireless transceiver; and
a handheld device comprising a processor, a wireless transceiver, a global positioning system, and a monitor, wherein the wireless transceiver of the handheld device exchanges data with the wireless transceiver of the pair of shoes wherein the monitor displays at least one of an amount of impacts detected by the impact detector, a location of the handheld device, a distance traveled of the handheld device, and a speed of the handheld device.

2. The workout system of claim 1, wherein the pair of shoes further comprise a light emitter, wherein the handheld device comprises a controller operable to turn the light emitter on and off.

3. The workout system of claim 1, wherein the handheld device further comprises a music player, a memory operable to store music data and an output connectable to head phones.

4. The workout system of claim 3, further comprising a wireless internet connection operable to download data to the memory.

5. The workout system of claim 1, further comprising a heart rate monitor operably connected to the processor, wherein the heart rate monitor sends heart rate data to the processor, which is displayed on the monitor.

6. The workout system of claim 1, wherein the handheld device further comprises a texting function.

7. The workout system of claim 1, wherein the handheld device further comprises an emergency function, wherein when activated the emergency function is operable to send a distress signal from the transceiver to a third party device.

8. The workout system of claim 1, wherein the monitor displays a time, a date, and a temperature.
